SEQUENCE LISTING (27 sequences)

SEQ ID NO: 1 - DNA, 19 bp, synthetic construct
  source 1..19: mol_type = other DNA
  misc_feature 1..19: note = sense primer
raaccaaaasgctccccrt

SEQ ID NO: 2 - DNA, 27 bp, synthetic construct
  source 1..27: mol_type = other DNA
  misc_feature 1..27: note = antisense primer
ttatatgtyggttacktgcgtttrtat

SEQ ID NO: 3 - DNA, 22 bp, synthetic construct
  source 1..22: mol_type = other DNA
  misc_feature 1..22: note = probe
yccrtcraaaacyygccgatta

SEQ ID NO: 4 - DNA, 19 bp, synthetic construct
  source 1..19: mol_type = other DNA
  misc_feature 1..19: note = sense primer
cggamgcgttcsggtraag

SEQ ID NO: 5 - DNA, 18 bp, synthetic construct
  source 1..18: mol_type = other DNA
  misc_feature 1..18: note = antisense primer
aattccrccgcsccraac

SEQ ID NO: 6 - DNA, 29 bp, synthetic construct
  source 1..29: mol_type = other DNA
  misc_feature 1..29: note = probe
tttccgckaaatatcktttcttcktcgca

SEQ ID NO: 7 - DNA, 24 bp, synthetic construct
  source 1..24: mol_type = other DNA
  misc_feature 1..24: note = sense primer
cscggtttggagttagagttatag

SEQ ID NO: 8 - DNA, 20 bp, synthetic construct
  source 1..20: mol_type = other DNA
  misc_feature 1..20: note = antisense primer
tacgaaacycgacgcataaa

SEQ ID NO: 9 - DNA, 24 bp, synthetic construct
  source 1..24: mol_type = other DNA
  misc_feature 1..24: note = probe
acgcgaaacmgatccgtaaactcc

SEQ ID NO: 10 - DNA, 20 bp, synthetic construct
  source 1..20: mol_type = other DNA
  misc_feature 1..20: note = sense primer
atygagtygcgggagttrgt

SEQ ID NO: 11 - DNA, 20 bp, synthetic construct
  source 1..20: mol_type = other DNA
  misc_feature 1..20: note = antisense primer
acacgckccaaccgartayg

SEQ ID NO: 12 - DNA, 18 bp, synthetic construct
  source 1..18: mol_type = other DNA
  misc_feature 1..18: note = probe
cccktcccaacgcsccya

SEQ ID NO: 13 - DNA, 22 bp, synthetic construct
  source 1..22: mol_type = other DNA
  misc_feature 1..22: note = sense primer
aggcgatsgtttrgtygtaagt

SEQ ID NO: 14 - DNA, 20 bp, synthetic construct
  source 1..20: mol_type = other DNA
  misc_feature 1..20: note = antisense primer
ccckactatcccgcsgatya

SEQ ID NO: 15 - DNA, 24 bp, synthetic construct
  source 1..24: mol_type = other DNA
  misc_feature 1..24: note = probe
ttcraatctcaacgaackcygcgc

SEQ ID NO: 16 - DNA, 20 bp, synthetic construct
  source 1..20: mol_type = other DNA
  misc_feature 1..20: note = sense primer
gtttcggtcrtsgagtttga

SEQ ID NO: 17 - DNA, 22 bp, synthetic construct
  source 1..22: mol_type = other DNA
  misc_feature 1..22: note = antisense primer
cctacgccsaacgatrctckat

SEQ ID NO: 18 - DNA, 25 bp, synthetic construct
  source 1..25: mol_type = other DNA
  misc_feature 1..25: note = probe
ttmgagaagtgtttcgagmgcgygg

SEQ ID NO: 19 - DNA, 21 bp, synthetic construct
  source 1..21: mol_type = other DNA
  misc_feature 1..21: note = sense primer
cggcggtagtmgtagtsgtag

SEQ ID NO: 20 - DNA, 21 bp, synthetic construct
  source 1..21: mol_type = other DNA
  misc_feature 1..21: note = antisense primer
gccscgatcgackaacraata

SEQ ID NO: 21 - DNA, 26 bp, synthetic construct
  source 1..26: mol_type = other DNA
  misc_feature 1..26: note = probe
tcsgagttcgtmgtagtttcgagmgt

SEQ ID NO: 22 - DNA, 20 bp, synthetic construct
  source 1..20: mol_type = other DNA
  misc_feature 1..20: note = sense primer
ygattggygggtgaygattt

SEQ ID NO: 23 - DNA, 21 bp, synthetic construct
  source 1..21: mol_type = other DNA
  misc_feature 1..21: note = antisense primer
accgtaackacaaacgckasg

SEQ ID NO: 24 - DNA, 29 bp, synthetic construct
  source 1..29: mol_type = other DNA
  misc_feature 1..29: note = probe
agttcgtattaaygagttggcggtttygt

SEQ ID NO: 25 - DNA, 27 bp, synthetic construct
  source 1..27: mol_type = other DNA
  misc_feature 1..27: note = sense primer
ggtggaggtagttagggtttatttgta

SEQ ID NO: 26 - DNA, 31 bp, synthetic construct
  source 1..31: mol_type = other DNA
  misc_feature 1..31: note = antisense primer
ccacaccacaaaatcacacttaacctcattt

SEQ ID NO: 27 - DNA, 21 bp, synthetic construct
  source 1..21: mol_type = other DNA
  misc_feature 1..21: note = probe
cacttttattcaactaatctc

(19) United States
(12) Patent Application Publication
(10) Pub. No.: US 2025/0329465 A1
(43) Pub. Date: Oct. 23, 2025

(54) METHOD OF DETECTING EARLY-STAGE HEPATOCELLULAR CARCINOMA

(71) Applicant: Yun Xiang Investment Consulting Co., Ltd., Hsinchu County (TW)

(72) Inventors: Chang-Yi LU, Hsinchu County (TW); Pey-Jey PENG, Hsinchu City (TW)

(21) Appl. No.: 19/176,583

(22) Filed: Apr. 11, 2025

Related U.S. Application Data
(60) Provisional application No. 63/637,109, filed on Apr. 22, 2024.

(51) Int. Cl. G16H 50/30; C12Q 1/6827; C12Q 1/6851; C12Q 1/686; C12Q 1/6886; G16B 20/20

(52) U.S. Cl. CPC G16H 50/30; C12Q 1/6827; C12Q 1/6851; C12Q 1/686; C12Q 1/6886; G16B 20/20; C12Q 2600/154; C12Q 2600/178

(57) ABSTRACT

A method of detecting early-stage hepatocellular carcinoma has steps of performing biomarker identification of a plurality of differentially methylated genes in a computing system; performing quantitative measurement of the methylation levels of the biomarkers selected from the group of differentially methylated genes with quantitative methylation-specific PCR in the computing system; performing calculation of a formula in the computing system to obtain M-score of the selected biomarkers according to the measured methylation levels of the selected biomarkers with a logistic regression analysis; and performing a risk level evaluation of liver cancer with the M-score of the selected biomarkers in the computing system.

### CROSS-REFERENCE TO RELATED APPLICATIONS

The present application claims the benefit of the U.S. provisional application Ser. No. 63/637,109, filed on Apr. 22, 2024, which is incorporated herein by reference in its entirety.

### REFERENCE TO AN ELECTRONIC SEQUENCE LISTING

The content of the electronic sequence listing (LCY24002USUP Seq.xml; Size: 35 kb; and Date of Creation: Mar. 17, 2025) is herein incorporated by reference in its entirety.

### TECHNICAL FIELD

The present invention pertains to a method of detecting early-stage hepatocellular carcinoma (HCC) by utilizing differentially methylated genes as biomarkers and measuring methylation levels of these biomarkers. Additionally, this invention proposes a methylation prediction system and a specifically developed detection kit for the detection of early-stage HCC.

### BACKGROUND

Hepatocellular carcinoma (HCC) is a major global health concern, ranking as the sixth most diagnosed cancer and the third leading cause of cancer-related deaths. This highly malignant tumor has a poor prognosis and a high mortality rate, with the number of annual deaths nearly matching the number of newly diagnosed cases. The incidence of HCC varies by geographic location, due to regional differences in exposure to hepatitis B virus (HBV) and hepatitis C virus (HCV). Additionally, cirrhosis from any cause, along with hepatitis virus infections, significantly increases the risk of developing HCC.

HCC shows significant resistance to chemotherapy, with no chemotherapeutic agents proving to improve overall survival rates. The only curative treatments available are surgical interventions, such as partial liver resection and liver transplantation. However, less than 30% of HCC patients qualify for surgery, primarily due to late-stage diagnosis and the presence of multiple lesions on a cirrhotic or fibrotic liver. Consequently, early detection of HCC is essential, as it allows for the application of effective treatments that can improve overall survival rates.

Serum alpha-fetoprotein (AFP) is the most used tumor marker for screening and monitoring HCC, despite its limitations in sensitivity and specificity. When using a cut-off value of 20 ng/mL, the reported sensitivities of AFP for HCC in cirrhotic patients range from 41% to 65% [1]. However, during the early stages of HCC progression, detection rates can be as low as one-third, since 80% of small HCC cases do not show elevated serum AFP levels [2,3]. In contrast, elevated AFP levels can also be observed in other chronic liver diseases, such as cirrhosis and hepatic inflammation, as well as in other types of cancer, including nonseminomatous germ cell tumors and gastrointestinal cancers [4]. Therefore, additional biomarkers are needed to supplement AFP to improve the accuracy of diagnosis, particularly for early-stage HCC.

Over the past few decades, DNA methylation has been increasingly recognized as a valuable biomarker for the early detection and diagnosis of cancer. DNA methylation is a key mechanism that regulates gene expression in normal cells and plays a significant role in many physiological processes. Abnormal DNA methylation can result in various human diseases, including cancers. DNA methyltransferases catalyze the addition of a methyl group to the carbon-5 position of cytosine residues in CpG dinucleotides. This methylation of the promoter or 5′ region of CpG islands can lead to the transcriptional repression of downstream genes. There is substantial evidence that DNA hypermethylation can downregulate tumor suppressors and DNA repair genes, while hypomethylation can upregulate oncogenes during the early stages of carcinogenesis [5,6]. DNA methylation involves the covalent binding of a methyl group to genomic DNA, making it more stable than protein or RNA markers. Furthermore, methylation markers can be detected in various types of liquid biopsies, such as blood, urine, saliva, and stool, providing a non-invasive method for monitoring cancer progression [7].

In our previous study, we employed a whole-genome approach to identify significant DNA methylation profiles in HCC cell lines and tissues [8-10]. We selected a panel of eight genes and miRNAs regulated by DNA methylation and measured their methylation levels in plasma cell-free DNA. The predictive capability of these markers for HCC diagnosis was evaluated both independently and in combination with the current HCC marker, AFP, to assess their potential for future clinical applications. Furthermore, we developed a methylation predictive system and kit specifically designed for the early diagnosis of HCC.

The following is a list of references that are occasionally cited in the above-mentioned description. The disclosures of these references are incorporated by reference herein in their entirety.
[1]. Um T-H, Kim H, Oh B-K, Kim M S, Kim K S, Jung G, et al. Aberrant CpG island hypermethylation in dysplastic nodules and early HCC of hepatitis B virus-related human multistep hepatocarcinogenesis. Journal of Hepatology. 2011; 54:939-47.
[2]. Lu C-Y, Hsieh S-Y, Lu Y-J, Wu C-S, Chen L-C, Lo S-J, et al. Aberrant DNA methylation profile and frequent methylation of KLK10 and OXGR1 genes in hepatocellular carcinoma. Genes Chromosom Cancer. 2009; 48:1057-68.
[3]. Kitamura Y, Shirahata A, Sakuraba K, Goto T, Mizukami H, Saito M, et al. Aberrant methylation of the Vimentin gene in hepatocellular carcinoma. Anticancer Res. 2011; 31:1289-91.
[4]. Zamcheck N, Pusztaszeri G. CEA, AFP and Other Potential Tumor Markers. CA: A Cancer Journal for Clinicians. 1975; 25:204-14.
[5]. Lu C-Y, Chen S-Y, Peng H-L, Kan P-Y, Chang W-C, Yen C-J. Cell-free methylation markers with diagnostic and prognostic potential in hepatocellular carcinoma. Oncotarget. 2017; 8:6406-18.
[6]. Samman B S, Hussein A, Samman R S, Alharbi A S. Common Sensitive Diagnostic and Prognostic Markers in Hepatocellular Carcinoma and Their Clinical Significance: A Review. Cureus [Internet]. 2022 [cited 2023 Sep 27]; Available from: https://www.cureus.com/articles/92250-common-sensitive-diagnostic-and-prognostic-markers-in-hepatocellular-carcinoma-and-their-clinical-significance-a-review
[7]. Debruyne E N, Delanghe J R. Diagnosing and monitoring hepatocellular carcinoma with alpha-fetoprotein: New aspects and applications. Clinica Chimica Acta. 2008; 395:19-26.
[8]. Widschwendter M, Apostolidou S, Raum E, Rothenbacher D, Fiegl H, Menon U, et al. Epigenotyping in Peripheral Blood Cell DNA and Breast Cancer Risk: A Proof of Principle Study. Goodyear M, editor. PLoS ONE. 2008; 3:e2656.
[9]. Lu C-Y, Lin K-Y, Tien M-T, Wu C-T, Uen Y-H, Tseng T-L. Frequent DNA methylation of MiR-129-2 and its potential clinical implication in hepatocellular carcinoma: miR-129-2 Hypermethylation in HCC. Genes Chromosomes Cancer. 2013; n/a-n/a.
[10]. Bibikova M, Barnes B, Tsan C, Ho V, Klotzle B, Le J M, et al. High density DNA methylation array with single CpG site resolution. Genomics. 2011; 98:288-95.

### SUMMARY

This invention presents a method for detecting and quantifying the methylation levels of genes and miRNAs through computational means, serving as biomarkers for detection of HCC. The assessment of these methylated gene biomarkers will enhance traditional prediction tools, enabling more precise detection of liver cancer at its early stages.

The present disclosure provides a method of detecting early-stage hepatocellular carcinoma (HCC) to evaluate the risk of suffering liver cancer in a subject. In one embodiment, the method includes the following steps: (a) performing biomarker identification of a group of differentially methylated genes in a computing system designed to process, analyze, simulate, and model biological data and equipped with a microprocessor by respectively detecting methylation level of the group of differentially methylated genes, the group of differentially methylated genes consists of APC gene, COX2 gene, miR-203, RASSF1A gene, VIM gene, RGS10 gene, ST8SIA6 gene, and miR-129-2, respectively, in bio-samples coming from the subject; (b) performing quantitative measurement of the methylation levels of a plurality of biomarkers selected from the group of differentially methylated genes with quantitative methylation-specific PCR (qMSP) in the computing system, wherein the selected biomarkers are the APC gene, COX2 gene, miR-203, RASSF1A gene, VIM gene, RGS10 gene, ST8SIA6 gene, and miR-129-2; (c) performing calculation of a formula in the computing system to obtain M-score of the selected biomarkers according to the methylation levels of the selected biomarkers with a logistic regression analysis, wherein the selected biomarkers are the APC gene, COX2 gene, miR-203, RASSF1A gene, VIM gene, RGS10 gene, ST8SIA6 gene, and miR-129-2; and performing a risk level evaluation of liver cancer with the M-score of the selected biomarkers in the computing system. The formula is M-score=X1+X2×ln(APC)+X3×ln(COX2)+X4×ln(miR-203)+X5×ln(RASSF1A)+X6×ln(VIM)+X7×ln(RGS10)+X8×ln(ST8SIA6)+X9×ln(miR-129-2), X1 ranges from 3.701 to 4.914, X2 ranges from 0.117 to 0.229, X3 ranges from 0.104 to 0.176, X4 ranges from 0.114 to 0.254, X5 ranges from 0.125 to 0.237, X6 ranges from 0.213 to 0.317, X7 ranges from 0.075 to 1.087, X8 ranges from 0.085 to 0.108, X9 ranges from 0.047 to 0.086, ln(APC) represents a hyperbolic logarithm of the methylation level of the APC gene, ln(COX2) represents a hyperbolic logarithm of the methylation level of the COX2 gene, ln(miR-203) represents a hyperbolic logarithm of the methylation level of the miR-203, ln(RASSF1A) represents a hyperbolic logarithm of the methylation level of the RASSF1A gene, ln(VIM) represents a hyperbolic logarithm of the methylation level of the VIM gene, ln(RGS10) represents a hyperbolic logarithm of the methylation level of the RGS10 gene, ln(ST8SIA6) represents a hyperbolic logarithm of the methylation level of the ST8SIA6 gene, and ln(miR-129-2) represents a hyperbolic logarithm of the methylation level of the miR-129-2.

In one embodiment, the method of detecting early-stage HCC further includes a step of performing a receiver operating characteristic (ROC) curve analysis of the M-score of the selected biomarkers in the computing system.

In one embodiment, the step of performing the quantitative measurement of the methylation levels of the selected biomarkers with the quantitative methylation-specific PCR (qMSP) in the computing system includes performing a calculation of a difference in Ct value between housekeeping gene and each of the selected biomarkers and a formula 2^{[Ct(housekeeping gene)-Ct(biomarker)]}×100 is used to perform the calculation.

In one embodiment, the housekeeping gene mentioned is a gene selected from a group consisting of β-actin, GAPDH, HPRT, YWHAZ, ARBP, SDHA and UBC.

In one embodiment, the step of performing the quantitative measurement of the methylation levels of the selected biomarkers with the quantitative methylation-specific PCR (qMSP) in the computing system includes using a kit to detect, the kit has a plurality of primer and probe sets targeting the APC gene, COX2 gene, miR-203, RASSF1A gene, VIM gene, RGS10 gene, ST8SIA6 gene, and miR-129-2; and a qMSP master mix having Taq DNA polymerase, dNTPs, MgCl2 and buffer.

In one embodiment, each of the primer and probe sets mentioned includes a sense primer, an antisense primer, and a probe separately having a sequence correspondingly associated with the targeted gene.

In one embodiment, the methylation level of the APC gene is calculated using formula 2^{[Ct(β-actin)-Ct(APC)]}×100, the methylation level of the COX2 gene is calculated using formula 2^{[Ct(β-actin)-Ct(Cox2)]}×100, the methylation level of the MIR-203 is calculated using formula 2^{[Ct(β-actin)-Ct(MIR-203)]}×100, the methylation level of the RASSF1A gene is calculated using formula 2^{[Ct(β-actin)-Ct(RASSF1A)]}×100, the methylation level of the VIM gene is calculated using formula 2^{[Ct(β-actin)-Ct(VIM)]}×100, the methylation level of the RGS10 gene is calculated using formula 2^{[Ct(β-actin)-Ct(RGS10)]}×100, the methylation level of the ST8SIA6 gene is calculated using formula 2^{[Ct(β-actin)-Ct(ST8SIA6)]}×100, and the methylation level of the miR-129-2 is calculated using formula 2^{[Ct(β-actin)-Ct(miR-129-2)]}×100.

In another aspect, a kit used in the method of detecting early-stage HCC is provided. The kit has a plurality of primer-pair and probe sets designed for the separate detection of methylation levels of APC gene, COX2 gene, miR-203, RASSF1A gene, VIM gene, RGS10 gene, ST8SIA6 gene, and miR-129-2. Each of the primer-pair and probe sets has a primer-pair and a probe.

Biomarkers regulated by DNA methylation were identified in a computing system as participating in the early occurrence of liver cancer and may potentially act as a powerful predictor for early diagnosis of liver cancer. The “M-score” calculated in a computing manner in the present invention has proven to be effective in predicting liver cancer patients. The M-score exceeds the predictive capabilities of serum markers.

This invention discloses the role of DNA methylation in early diagnosis of live cancer. Some of the biomarkers were differentially methylated between liver cancer patients and non-tumor control, and these biomarkers could be used to calculate the “M-score” for liver cancer early diagnosis. This invention compared M-score with other clinical factors, such as AFP, and found that M-score had a better performance in early diagnosis of liver cancer.

Various other objects, advantages, and features of the present invention will become readily apparent from the ensuing detailed description accompanying drawings, and the novel features will be particularly pointed out in the appended claims.

### BRIEF DESCRIPTION OF DRAWINGS

The following detailed descriptions, given by way of example, and not intended to limit the present invention solely thereto, will be best understood in conjunction with the accompanying figures.

FIGS. 1A to 1H illustrate the methylation levels of eight selected biomarkers in HCC and normal controls including healthy donors, hepatitis, and cirrhosis patients. The methylation levels were transformed by the natural logarithm. A paired t-test was used to determine statistical significance.

FIG. 2A illustrates receiver operator characteristic (ROC) curves for the M-score of the eight selected biomarkers including APC gene, COX2 gene, miR-203, RASSF1A gene, VIM gene, RGS10 gene, ST8SIA6 gene, and miR-129-2 (abbreviated as MPM-8G) in the receiver operating characteristic curve (ROC) analysis.

FIG. 2B illustrates receiver operator characteristic (ROC) curves for AFP at the cut-off value of 4.5 ng/mL in the receiver operating characteristic curve (ROC) analysis.

FIG. 2C illustrates receiver operator characteristic (ROC) curves for AFP at the cut-off value of 20 ng/mL in the receiver operating characteristic curve (ROC) analysis.

FIG. 2D illustrates receiver operator characteristic (ROC) curves for a combination of AFP and the M-score of the eight selected biomarkers (abbreviated as MPM-8G+AFP) in the receiver operating characteristic curve (ROC) analysis.

FIG. 3A illustrates the distribution of the M-scores of the eight selected biomarkers (abbreviated as MPM-8G) in healthy donors, hepatitis, cirrhosis, and HCC patients. The cut-off value is 0.478.

FIG. 3B illustrates the distribution of AFP value in healthy donors, hepatitis, cirrhosis, and HCC patients. The cut-off values for AFP at 4.5 and 20 ng/mL are indicated by the lower and upper horizontal lines, respectively.

FIG. 3C illustrates the distribution of the combination of AFP and the M-scores of the eight selected biomarkers (abbreviated as MPM-8G+AFP) in healthy donors, hepatitis, cirrhosis, and HCC patients. The cut-off value is 0.424.

FIG. 4A illustrates the distribution of the M-scores of the eight selected biomarkers (abbreviated as MPM-8G) in different stages of HCC. The X-axis represents the TNM staging classification. The cut-off values are indicated by the horizontal line.

FIG. 4B illustrates the distribution of AFP in different stages of HCC. The AFP value was transformed by natural logarithm. The X-axis represents the TNM staging classification. The cut-off values are indicated by the horizontal line.

FIG. 4C illustrates the distribution of the combination of the M-scores of the eight selected biomarkers and AFP (abbreviated as MPM-8G+AFP) in different stages of HCC. The X-axis represents the TNM staging classification. The cut-off values are indicated by the horizontal line.

FIG. 5 is a flow chart illustrating a method of detecting early-stage hepatocellular carcinoma (HCC) according to one embodiment of the present invention.

### DETAILED DESCRIPTION OF THE EMBODIMENTS

In the following description, the biomarker/biomarkers, and the corresponding embodiments of the detection/validation/identification/quantification methods are set forth as preferred examples. It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

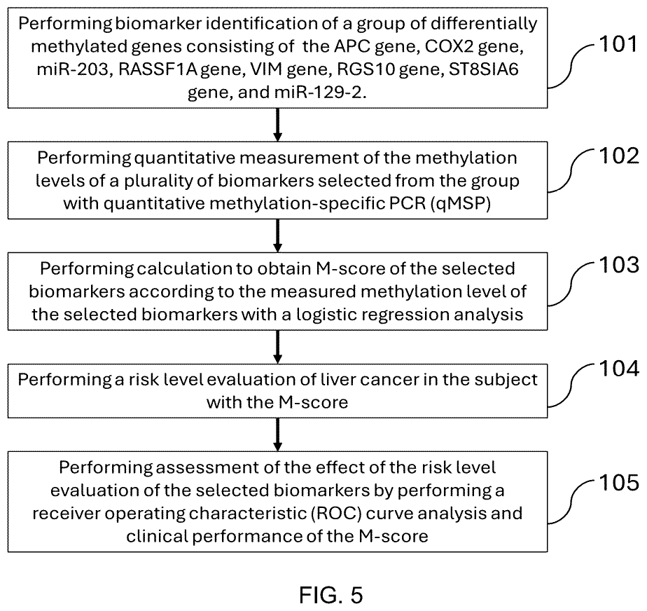

In one embodiment, a method of detecting early-stage HCC used to evaluate the risk of suffering from liver cancer in a subject may include, but is not limited to, the following step 101 to step 105, as shown in FIG. 5. The subject may include, but is not limited to, a mammal, such as a human, ape, monkey, cat, dog, rabbit, guinea pig, rat, or mouse. In one embodiment, the subject is for humans.

Step 101: performing biomarker identification of a group of differentially methylated genes consisting of the APC gene, COX2 gene, miR-203, RASSF1A gene, VIM gene, RGS10 gene, ST8SIA6 gene, and miR-129-2 by detecting the methylation levels or status of these differentially methylated genes and/or miRNAs, respectively, in bio-samples coming from the subject. The bio-sample may include, but is not limited to, blood, plasma, serum, liver tissue, saliva, sputum, semen, intestinal digestive, respiratory lavage, and feces. In one embodiment, the bio-sample is plasma or serum. The above-mentioned genes and/or miRNAs are used as potential biomarkers for early detection of HCC in the present invention.

Step 101 is preferably performed in a computing system, such as a computer, or a system equipped with a microprocessor capable of computing data and processing bio-signals. The computing system is designed to process, analyze, simulate, and model biological data and may employ specialized algorithms, software, and high-performance hardware to study and solve complex problems in biology, bioinformatics, computational biology, and other life science domains. Common applications of the computing system include genome sequencing and assembly, molecular modeling and simulation, bioinformatics databases, gene expression analysis, protein structure prediction, and drug design. Key components that enable the computing system to manage big biological data include clustering algorithms, artificial intelligence/machine learning models, distributed storage, and cloud computing, as well as advanced visualization capabilities. In one example, the computing system may include a biomaterial input device, a biomaterial processing unit, a bio-signal detection unit, and a data processing unit incorporating a microprocessor.

Step 102: performing quantitative measurement of the methylation levels of a plurality of biomarkers selected from the group of differentially methylated genes and/or miRNAs with quantitative methylation-specific PCR (qMSP) in the computing system. In one example, the selected biomarkers are the APC gene, COX2 gene, miR-203, RASSF1A gene, VIM gene, RGS10 gene, ST8SIA6 gene, and miR-129-2.

Step 103: performing calculation using a formula with a logistic regression analysis in the computing system to obtain an M-score of the selected biomarkers according to the measured methylation levels or status of the selected biomarkers. In one example, the selected biomarkers are APC gene, COX2 gene, miR-203, RASSF1A gene, VIM gene, RGS10 gene, ST8SIA6 gene, and miR-129-2.

Step 104: performing a risk level evaluation of liver cancer in the subject with the M-score or the combination of M-score and AFP in the computing system. In one embodiment, the subject has the risk of being afflicted with liver cancer when the M-score or the combination of M-score and AFP is higher compared to a pre-confirmed reference value.

In one embodiment, the method of detecting early-stage HCC used to evaluate the risk of suffering liver cancer in the subject may further include the following step 105, as shown in FIG. 5.

Step 105: performing assessment of the effect of the risk level evaluation of the selected biomarkers by performing a receiver operating characteristic (ROC) curve analysis of the M-score or the combination of M-score and AFP in the computing system.

In step 101, the detection of the methylation levels or status of APC gene, COX2 gene, miR-203, RASSF1A gene, VIM gene, RGS10 gene, ST8SIA6 gene, and miR-129-2 may utilize analysis methods include, but is not limited to, quantitative methylation-specific polymerase chain reaction (quantitative methylation-specific PCR, qMSP), combined bisulfite restriction analysis (COBRA), Bisulfite Sequencing, Pyrosequencing, Next Generation sequencing (NGS), and DNA Methylation Array Chip Analysis. The analysis methods are performed in the computing system.

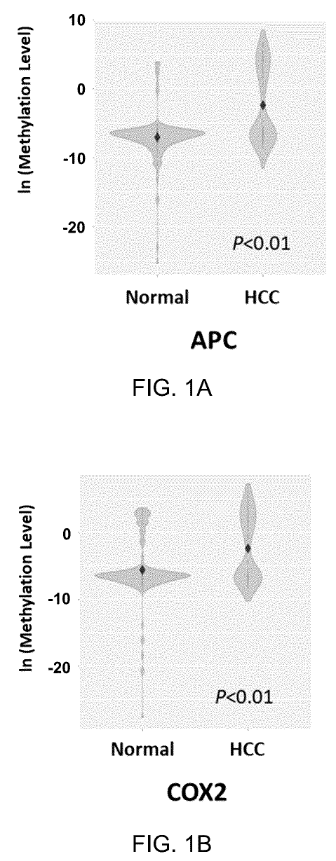

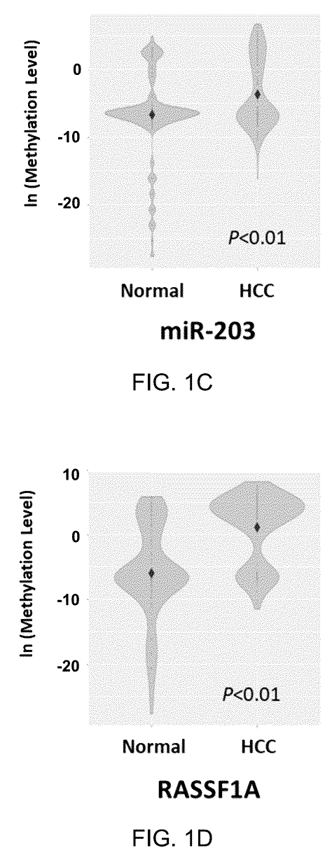

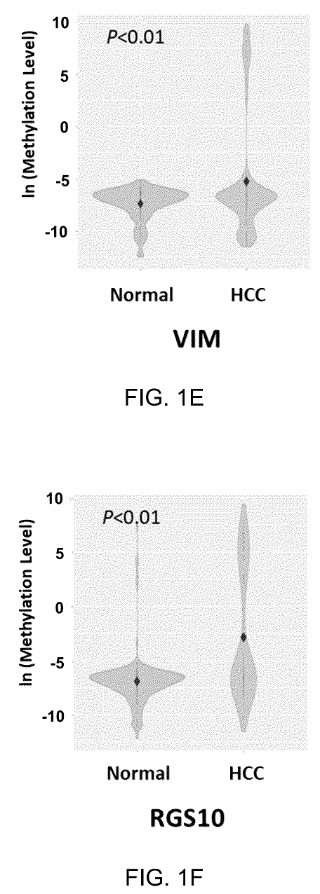

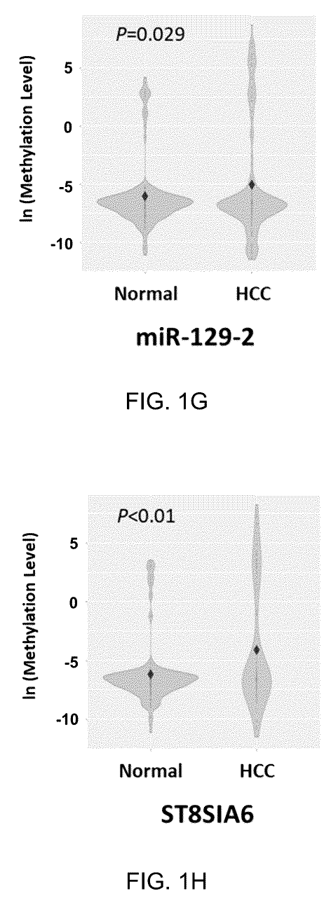

In one embodiment, the detection of the methylation levels or status of APC gene, COX2 gene, miR-203, RASSF1A gene, VIM gene, RGS10 gene, ST8SIA6 gene, and miR-129-2 in step 101 utilizes the quantitative methylation-specific PCR. In one example, the bio-samples are 318 plasma samples coming from 159 normal control and 159 HCC patients as shown in Table 1. The 318 plasma samples include healthy donors (n=52), chronic hepatitis B (n=61), chronic hepatitis B with cirrhosis (n=46), and HBV-related HCC (n=159). Referring to FIG. 1A to FIG. 1H, APC gene, COX2 gene, miR-203, RASSF1A gene, VIM gene, RGS10 gene, ST8SIA6 gene, and miR-129-2 showed higher methylation level in HCC patients than in normal control, with statistically significant differences, i.e., the P value is less than 0.05. As a result, the APC gene, COX2 gene, miR-203, RASSF1A gene, VIM gene, RGS10 gene, ST8SIA6 gene, and miR-129-2 may be considered biomarkers for HCC diagnosis.

TABLE 1

| | | Healthy Donor | Hepatitis | Cirrhosis | HCC |
|---|---|---|---|---|---|
| | | (N = 52) | (N = 61) | (N = 46) | (N = 159) |
| Age, median (range) | | 58 (29-78) | 46 (24-84) | 57.5 (26-92) | 61 (32-94) |
| Gender | Male | 34 | 43 | 36 | 131 |
| | Female | 17 | 18 | 10 | 28 |
| TNM | I | — | — | — | 24 |
| classification | II | — | — | — | 29 |
| | III | — | — | — | 11 |
| AFP | median, | 2.38 | 2.43 | 3.25 | 3.84 |
| | ng/mL | | | | |
| | >20, ng/ | 0 | 0 | 2.2 | 27.8 |
| | mL (%) | | | | |

In one embodiment, the detection of the methylation levels or status of APC gene may use a primer-pair including a sense primer, and an antisense primer, and a probe, wherein the sense primer has a sequence of at least 85% sequence similarity to SEQ ID NO: 1 as listed in Table 2, the antisense primer has a sequence of at least 85% sequence similarity to SEQ ID NO: 2 as listed in Table 2, and the probe has a sequence of at least 85% sequence similarity to SEQ ID NO: 3 as listed in Table 2. In one embodiment, the detection of the methylation level or status of COX2 gene may use a primer-pair including a sense primer, and an antisense primer, and a probe, wherein the sense primer has a sequence of at least 85% sequence similarity to SEQ ID NO: 4 as listed in Table 2, the antisense primer has a sequence of at least 85% sequence similarity to SEQ ID NO: 5 as listed in Table 2, and the probe has a sequence of at least 85% sequence similarity to SEQ ID NO: 6 as listed in Table 2. In one embodiment, the detection of the methylation level or status of miR-203 may use a primer-pair including a sense primer, and an antisense primer, and a probe, wherein the sense primer has a sequence of at least 85% sequence similarity to SEQ ID NO: 7 as listed in Table 2, the antisense primer has a sequence of at least 85% sequence similarity to SEQ ID NO: 8 as listed in Table 2, and the probe has a sequence of at least 85% sequence similarity to SEQ ID NO: 9 as listed in Table 2. In one embodiment, the detection of the methylation level or status of RASSF1A gene may use a primer-pair including a sense primer, and an antisense primer, and a probe, wherein the sense primer has a sequence of at least 85% sequence similarity to SEQ ID NO: 10 as listed in Table 2, the antisense primer has a sequence of at least 85% sequence similarity to SEQ ID NO: 11 as listed in Table 2, and the probe has a sequence of at least 85% sequence similarity to SEQ ID NO: 12 as listed in Table 2. In one embodiment, the detection of the methylation level or status of VIM gene may use a primer-pair including a sense primer, and an antisense primer, and a probe, wherein the sense primer has a sequence of at least 85% sequence similarity to SEQ ID NO: 13 as listed in Table 2, the antisense primer has a sequence of at least 85% sequence similarity to SEQ ID NO: 14 as listed in Table 2, and the probe has a sequence of at least 85% sequence similarity to SEQ ID NO: 15 as listed in Table 2. In one embodiment, the detection of the methylation level or status of RGS10 gene may use a primer-pair including a sense primer, and an antisense primer, and a probe, wherein the sense primer has a sequence of at least 85% sequence similarity to SEQ ID NO: 16 as listed in Table 2, the antisense primer has a sequence of at least 85% sequence similarity to SEQ ID NO: 17 as listed in Table 2, and the probe has a sequence of at least 85% sequence similarity to SEQ ID NO: 18 as listed in Table 2. In one embodiment, the detection of the methylation level or status of ST8SIA6 gene may use a primer-pair including a sense primer, and an antisense primer, and a probe, wherein the sense primer has a sequence of at least 85% sequence similarity to SEQ ID NO: 19 as listed in Table 2, the antisense primer has a sequence of at least 85% sequence similarity to SEQ ID NO: 20 as listed in Table 2, and the probe has a sequence of at least 85% sequence similarity to SEQ ID NO: 21 as listed in Table 2. In one embodiment, the detection of the methylation level or status of miR-129-2 may use a primer-pair including a sense primer, and an antisense primer, and a probe, wherein the sense primer has a sequence of at least 85% sequence similarity to SEQ ID NO: 22 as listed in Table 2, the antisense primer has a sequence of at least 85% sequence similarity to SEQ ID NO: 23 as listed in Table 2, and the probe has a sequence of at least 85% sequence similarity to SEQ ID NO: 24 as listed in Table 2.

In one embodiment, APC gene, COX2 gene, miR-203, RASSF1A gene, VIM gene, RGS10 gene, ST8SIA6 gene, and miR-129-2 are chosen as the selected biomarkers in step 102. Moreover, a primer-pair including a sense primer and antisense primer, and a probe correspondingly listed in Table 2 for APC gene, COX2 gene, miR-203, RASSF1A gene, VIM gene, RGS10 gene, ST8SIA6 gene, and miR-129-2 are used in the detection.

The usage of the quantitative methylation-specific PCR and the primer-pair to quantitatively measure the methylation levels or status of a methylated gene are described in the following examples.

In one example, a cell-free DNA from 400 ml plasma of a subject was bisulfite-converted by treatment of sodium bisulfite. The bisulfite-converted cell-free DNA was amplified by real-time quantitative methylation-specific PCR (qMSP) using fluorescent probes. Each reaction involved 1×qPCR Master Mix, 0.5 μM of each primer and 0.25 μM of probe in a total volume of 20 μl. Amplification was performed on the StepOnePlus Real-Time PCR System (Thermo Fisher Scientific). The methylation level of a selected biomarker of a methylated gene may be calculated from the formula: 2^{[Ct(housekeeping gene)-Ct(biomarker)]}×100, wherein Ct is the value of single data points derived from real-time PCR amplification plots, and the housekeeping gene may be a gene selected from a group consisting of β-actin, GAPDH, HPRT, YWHAZ, ARBP, SDHA and UBC. In one example, the methylation level was calculated as the difference in Ct value between beta-actin and the selected biomarkers using the following formula: 2^{[Ct(beta-actin)-Ct(biomarkers)]}×100. In one example, the primer-pairs and probe utilized in detecting methylation levels of the APC gene, COX2 gene, miR-203, RASSF1A gene, VIM gene, RGS10 gene, ST8SIA6 gene, and miR-129-2 are illustrated as follows.

TABLE 2

| Target | Primer/ | |
|---|---|---|
| gene | Probe | Sequence |
| APC | Sense primer | RAACCAAAASGCTCCCCRT |
| | | (SEQ ID No. 1) |
| | Antisense | TTATATGTYGGTTACKTGCGTTTR |
| | primer | TAT |
| | | (SEQ ID No. 2) |
| | Probe | YCCRTCRAAAACYYGCCGATTA |
| | | (SEQ ID No. 3) |
| COX2 | Sense primer | CGGAMGCGTTCSGGTRAAG |
| | | (SEQ ID No. 4) |
| | Antisense | AATTCCRCCGCSCCRAAC |
| | primer | (SEQ ID No. 5) |
| | Probe | TTTCCGCKAAATATCKTTTCTTCK |
| | | TCGCA |
| | | (SEQ ID No. 6) |
| miR-203 | Sense primer | CSCGGTTTGGAGTTAGAGTTATAG |
| | | (SEQ ID No. 7) |
| | Antisense | TACGAAACYCGACGCATAAA |
| | primer | (SEQ ID No. 8) |
| | Probe | ACGCGAAACMGATCCGTAAACTCC |
| | | (SEQ ID No. 9) |
| RASSF1A | Sense primer | ATYGAGTYGCGGGAGTTRGT |
| | | (SEQ ID No. 10) |
| | Antisense | ACACGCKCCAACCGARTAYG |
| | primer | (SEQ ID No. 11) |
| | Probe | CCCKTCCCAACGCSCCYA |
| | | (SEQ ID No. 12) |
| VIM | Sense primer | AGGCGATSGTTTRGTYGTAAGT |
| | | (SEQ ID No. 13) |
| | Antisense | CCCKACTATCCCGCSGATYA |
| | primer | (SEQ ID No. 14) |
| | Probe | TTCRAATCTCAACGAACKCYGCGC |
| | | (SEQ ID No. 15) |
| RGS10 | Sense primer | GTTTCGGTCRTSGAGTTTGA |
| | | (SEQ ID No. 16) |
| | Antisense | CCTACGCCSAACGATRCTCKAT |
| | primer | (SEQ ID No. 17) |
| | Probe | TTMGAGAAGTGTTTCGAGMGCGYG |
| | | G |
| | | (SEQ ID No. 18) |
| ST8SIA6 | Sense primer | CGGCGGTAGTMGTAGTSGTAG |
| | | (SEQ ID No. 19) |
| | Antisense | GCCSCGATCGACKAACRAATA |
| | primer | (SEQ ID No. 20) |
| | Probe | TCSGAGTTCGTMGTAGTTTCGAGM |
| | | GT |
| | | (SEQ ID No. 21) |
| miR-129-2 | Sense primer | YGATTGGYGGGTGAYGATTT |
| | | (SEQ ID No. 22) |
| | Antisense | ACCGTAACKACAAACGCKASG |
| | primer | (SEQ ID No. 23) |
| | Probe | AGTTCGTATTAAYGAGTTGGCGGT |
| | | TTYGT |
| | | (SEQ ID No. 24) |
| β-actin | Sense primer | GGTGGAGGTAGTTAGGGTTTATTT |
| | | GTA |
| | | (SEQ ID No. 25) |
| | Antisense | CCACACCACAAAATCACACTTAAC |
| | primer | CTCATTT |
| | | (SEQ ID No. 26) |
| | Probe | CACTTTTATTCAACTAATCTC |
| | | (SEQ ID No. 27) |

In one embodiment, the step 103 is performed to obtain the M-score of the selected biomarkers according to the methylation levels or status of the selected biomarkers being the APC gene, COX2 gene, miR-203, RASSF1A gene, VIM gene, RGS10 gene, ST8SIA6 gene, and miR-129-2 by calculating the following formula:

$$M - score = X 1 + X 2 \times ln ( APC ) + X 3 \times ln ( COX 2 ) + X 4 \times ln ( miR - 203 ) + X 5 \times ln ( RASSF 1 A ) + X 6 \times ln ( VIM ) + X 7 \times ln ( RGS 10 ) + X 8 \times ln ( ST 8 SI A 6 ) + X 9 \times ln ( miR - 129 - 2 )$$

In the above formula, X1 ranges from 3.701 to 4.914, X2 ranges from 0.117 to 0.229, X3 ranges from 0.104 to 0.176, X4 ranges from 0.114 to 0.254, X5 ranges from 0.125 to 0.237, X6 ranges from 0.213 to 0.317, X7 ranges from 0.075 to 1.087, X8 ranges from 0.085 to 0.108, X9 ranges from 0.047 to 0.086. In the above formula, the “In (APC)” represents a hyperbolic logarithm of the methylation level of the APC gene, and the methylation level of the APC gene is calculated using the formula: 2^{[Ct(β-actin)-Ct(APC)]}×100. In the above formula, the “In (COX2)” represents a hyperbolic logarithm of the methylation level of the COX2 gene, and the methylation level of the COX2 gene is calculated using the formula: 2^{[Ct(β-actin)-Ct(Cox2)]}×100. In the above formula, the “In (miR-203)” represents a hyperbolic logarithm of the methylation level of the miR-203, and the methylation level of the miR-203 is calculated using the formula: 2^{[Ct(β-actin)-Ct(miR-203)]}×100. In the above formula, the “In (RASSF1A)” represents a hyperbolic logarithm of the methylation level of the RASSF1A gene, and the methylation level of the RASSF1A gene is calculated using the formula: 2^{[Ct(β-actin)-Ct(RASSF1A)]}×100. In the above formula, the “In (VIM)” represents a hyperbolic logarithm of the methylation level of the VIM gene, and the methylation level of the VIM gene is calculated using the formula: 2^{[Ct(β-actin)-Ct(VIM)]}×100. In the above formula, the “In (RGS10)” represents a hyperbolic logarithm of the methylation level of the RGS10 gene, and the methylation level of the RGS10 gene is calculated using the formula: 2^{[Ct(β-actin)-Ct(RGS10)]}×100. In the above formula, the “In (ST8SIA6)” represents a hyperbolic logarithm of the methylation level of the ST8SIA6 gene, and the methylation level of the ST8SIA6 gene is calculated using the formula: 2^{[Ct(β-actin)-Ct(ST8SIA6)]}×100. In the above formula, the “In (miR-129-2)” represents a hyperbolic logarithm of the methylation level of the miR-129-2, and the methylation level of the miR-129-2 is calculated using the formula: 2^{[Ct(β-actin)-Ct(miR-129-2)]}×100. In one example, M score=4.802+0.128×methylation level of ln(APC)+0.154×methylation level of ln(COX2)+0.116×methylation level of ln(miR203)+0.148×methylation level of ln(RASSF1A)+0.257×methylation level of ln(VIM)+0.088×methylation level of ln(RGS10)+0.082×methylation level of ln(ST8SIA6)+0.059×methylation level of ln(miR-129-2). Among the formulas, Ct is the value of single data points derived from real-time PCR amplification plots.

In one embodiment, the M-score with which step 104 is performed is calculated according to the methylation levels of the APC gene, COX2 gene, miR-203, RASSF1A gene, VIM gene, RGS10 gene, ST8SIA6 gene, and miR-129-2. In one example, the subject has the risk of afflicting liver cancer when the M-score or the combination of M-score and AFP is higher compared to a pre-confirmed reference value. In other words, the risk of being afflicted with liver cancer increases with the M-score compared to the pre-confirmed reference value.

In one embodiment, the step 104 may include a step of determining the pre-confirmed reference value by respectively comparing the methylation level of the APC gene, COX2 gene, miR-203, RASSF1A gene, VIM gene, RGS10 gene, ST8SIA6 gene, and miR-129-2 in one group of the subjects known not to have liver cancer with another group known to have liver cancer and obtaining a cutoff value from the receiver operating characteristic (ROC) curves in the receiver operating characteristic analysis performed in the computing system. In one example, the pre-confirmed reference value is 0.478 and the subject has the risk of afflicting with liver cancer when the M-score is higher than 0.478.

In one embodiment, the step 104 may include a step of determining the pre-confirmed reference value by respectively comparing the methylation level of the APC gene, COX2 gene, miR-203, RASSF1A gene, VIM gene, RGS10 gene, ST8SIA6 gene, miR-129-2 and AFP in one group of the subjects known not to have liver cancer with another group known to have liver cancer and obtaining a cutoff value from the receiver operating characteristic (ROC) curves in the receiver operating characteristic analysis performed in the computing system. In one example, the pre-confirmed reference value is 0.424 and the subject has the risk of afflicting with liver cancer when the M-score is higher than 0.424.

Given the above, in one embodiment, a kit made of a plurality of primer and probe sets of methylation biomarkers and a qPCR master mix may be preferably used to detect the biomarker methylation in the early diagnosis of liver cancer, wherein the primer and probe sets may respectively targe the APC gene (SEQ ID No.1-3 of Table 2), COX2 gene (SEQ ID No.4-6 of Table 2), miR-203 (SEQ ID No.7-9 of Table 2), RASSF1A gene (SEQ ID No.10-12 of Table 2), VIM gene (SEQ ID No.13-15 of Table 2), RGS10 gene (SEQ ID No.16-18 of Table 2), ST8SIA6 gene (SEQ ID No.19-21 of Table 2), miR-129-2 (SEQ ID No.22-24 of Table 2), and β-actin gene (SEQ ID No.25-27 of Table 2), and the qPCR master mix may have Taq DNA polymerase, dNTPs, MgCl2 and buffer.

In one embodiment, the step 105 carries out a receiver operating characteristic (ROC) curve analysis to assess the performance or the diagnostic effect of the methylation biomarkers including the APC gene, COX2 gene, miR-203, RASSF1A gene, VIM gene, RGS10 gene, ST8SIA6 gene, and miR-129-2.

In one embodiment, the step 105 carries out a receiver operating characteristic (ROC) curve analysis to assess the performance or the diagnostic effect of the methylation biomarkers including the APC gene, COX2 gene, miR-203, RASSF1A gene, VIM gene, RGS10 gene, ST8SIA6 gene, miR-129-2, and AFP.

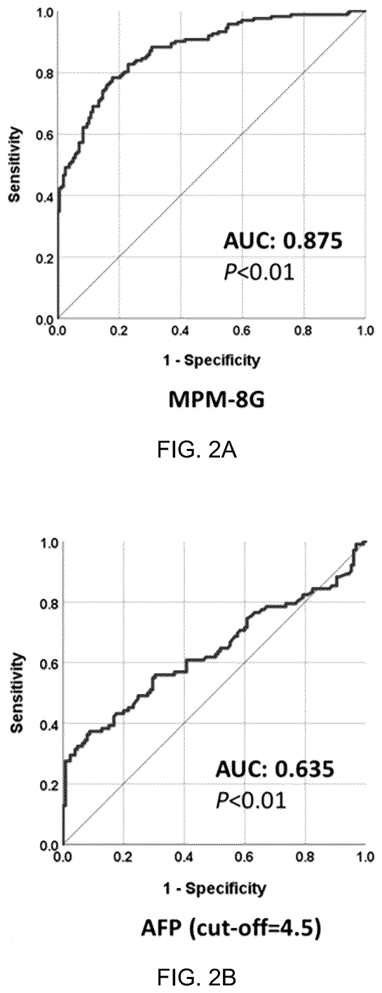

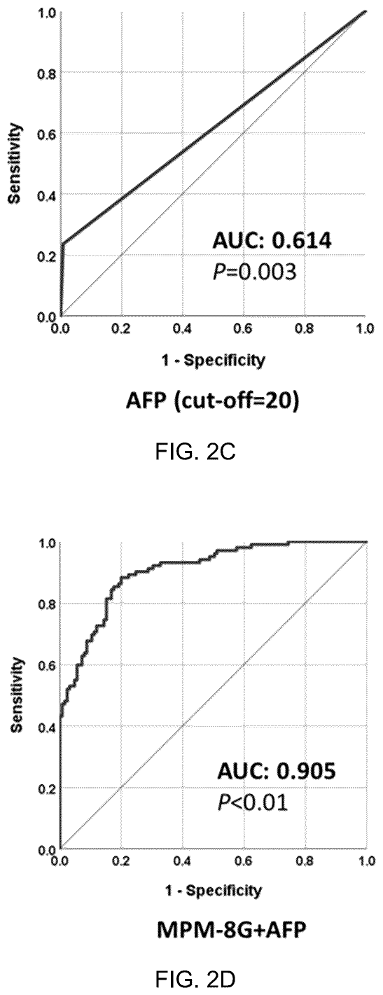

FIG. 2A illustrates receiver operator characteristic (ROC) curves for the M-score of the eight selected biomarkers including the APC gene, COX2 gene, miR-203, RASSF1A gene, VIM gene, RGS10 gene, ST8SIA6 gene, and miR-129-2 in the receiver operating characteristic curve (ROC) analysis. FIG. 2B illustrates receiver operator characteristic (ROC) curves for AFP (alpha-fetoprotein, cut-off at 4.5 ng/mL) in the receiver operating characteristic curve (ROC) analysis. FIG. 2C illustrates receiver operator characteristic (ROC) curves for AFP (alpha-fetoprotein, cut-off at 20 ng/mL) in the receiver operating characteristic curve (ROC) analysis. FIG. 2D illustrates receiver operator characteristic (ROC) curves for the combination of AFP and M-score of the eight selected biomarkers including the APC gene, COX2 gene, miR-203, RASSF1A gene, VIM gene, RGS10 gene, ST8SIA6 gene, and miR-129-2. The AUC (area under the curve) in view of the M-score in FIG. 2A is 0.875 (P<0.01). The AUC in view of AFP with cut-off at 4.5 ng/mL in FIG. 2B is 0.635 (P<0.01). The AUC in view of AFP with cut-off at 20 ng/mL in FIG. 2C is 0.614 (P=0.003). The AUC in view of the combination of M-score and AFP in FIG. 2D is 0.905 (P<0.01).

As shown in Table 3, the receiver operating characteristic (ROC) analysis determines the sensitivity (Sen), specificity (Spe), positive predictive value (PPV), negative predictive value (NPV), accuracy (ACC), false positive (FP), and false negative (FN) for the M-score, AFP and the combination of M-score and AFP, respectively. AFP shows excellent specificity and positive predictive value at the cut-off value of 20 ng/mL. However, its sensitivity is only 23.5%. The optimal cut-off value of AFP in our study is 4.5 ng/mL, elevating the sensitivity to 43.1%, which is still unacceptable for diagnosis. M-score shows around 80% of Sen, Spe, PPV, NPV, ACC, and around 20% of FP and FN. By combination of AFP and M-score, diagnostic ability makes huge progress in Sen (86.3%), NPV (87.8%), and FN (12.2%). These results showed the superior performance of M-score over the current serum tumor marker, and that the integration of M-score and AFP provides more accurate detection ability for HCC diagnosis.

TABLE 3

| | AFP (%) | AFP (%) | M-score (%) | M-score + AFP (%) |
|---|---|---|---|---|
| cut-off | 20 | 4.5 | 0.478 | 0.424 |
| SEN | 23.5 | 43.1 | 80.1 | 86.3 |
| SPE | 99.2 | 82.4 | 78.3 | 80.8 |
| PPV | 96 | 66.7 | 79.1 | 78.6 |
| NPV | 61.4 | 64.0 | 79.4 | 87.8 |
| ACC | 65.2 | 64.8 | 79.2 | 83.3 |
| FP | 4 | 33.3 | 22.2 | 21.4 |
| FN | 38.6 | 36.0 | 20.6 | 12.2 |

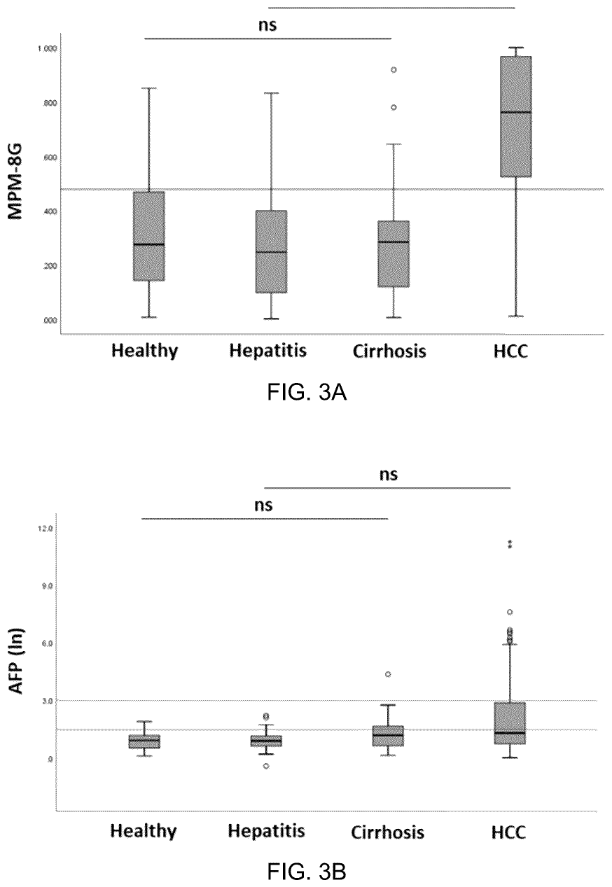

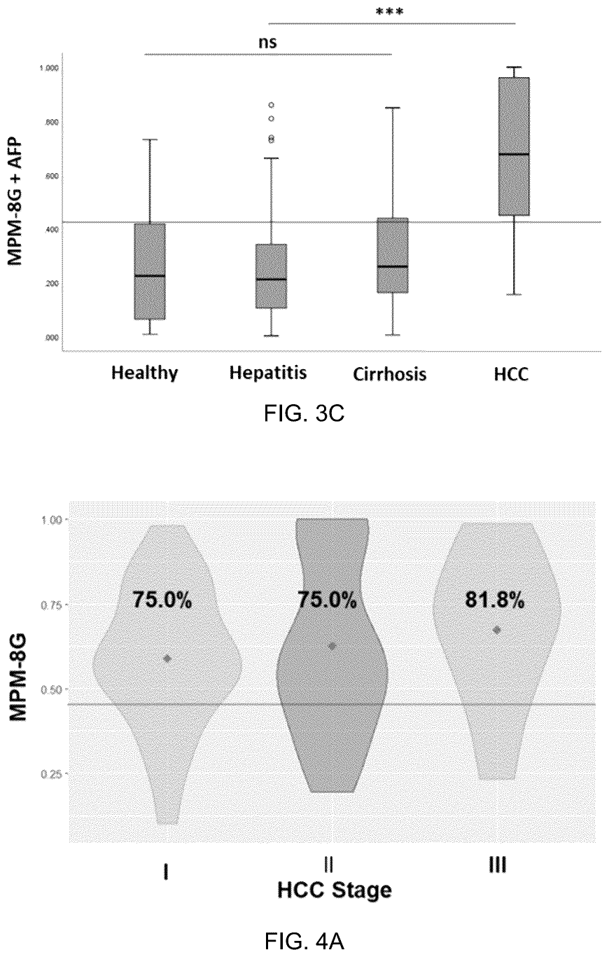

To further evaluate the specificity of M-score, the M-score and AFP level in HCC and non-tumor controls were examined, including healthy donors, hepatitis, and cirrhosis. The AFP value was transformed into a natural logarithm before analysis. The median AFP levels for healthy donors, hepatitis, cirrhosis, and HCC are 0.8961, 0.8755, 1.1663, and 1.2892, respectively. There is no significant difference in AFP levels between tumor and non-tumor controls (FIG. 3B). Regardless of the cut-off value used at 4.5 or 20 ng/mL, the median AFP of HCC patients remained below the cut-off value, indicating that over 50% of HCC patients could not be detected using any AFP cut-off values. On the other hand, M-score is significantly higher in HCC patients (median, 0.7613) compared to non-tumor controls (median values: healthy donors: 0.2745, hepatitis: 0.2173, and cirrhosis: 0.2833) (FIG. 3A). Combining M-score and AFP shows a superior ability to differentiate between non-tumor and HCC patients (FIG. 3C).

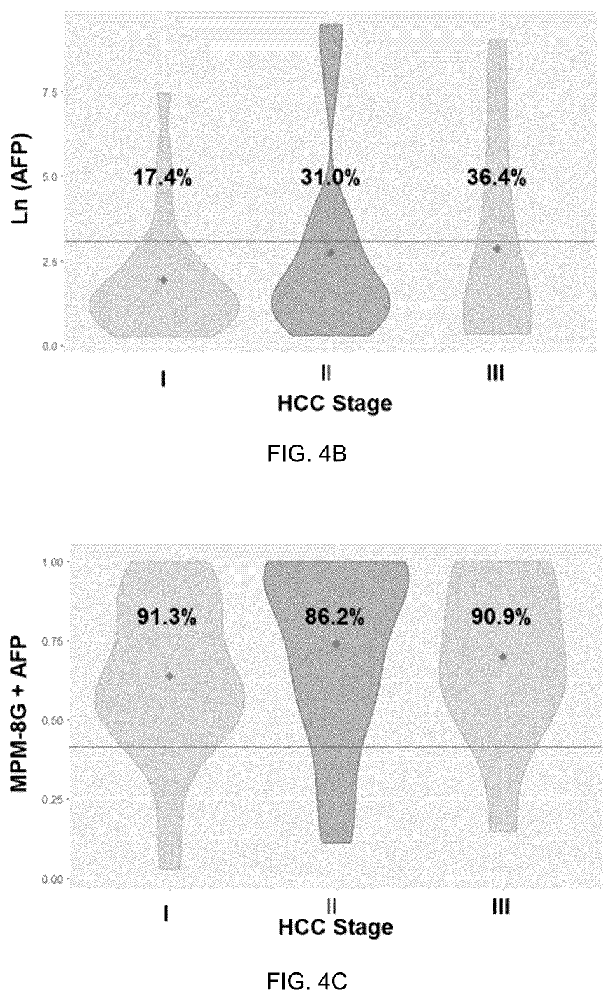

The detection rate of early stage of HCC was evaluated by M-score and AFP. Based on the cut-off value of M-score, the detection rates of M-score in stage I, II, and III are 75.0%, 75.0%, and 81.8%, respectively (FIG. 4A). In contrast, the detection rates of AFP were from 17.4% in stage I to 36.4% in stage III (FIG. 4B). Despite the stage-dependent manner AFP showed, the detection rate remains significantly low among patients in the first stage of HCC. In combination with M-score and AFP, the detection rate can be elevated to 91.3% in stage I, 86.2% in stage II, and 90.9% in stage III (FIG. 4C). These results indicate the combination of M-score and AFP may act as a very powerful tool for HCC early diagnosis.

Biomarkers regulated by DNA methylation were identified in a computing system as participating in the early diagnosis of liver cancer and may potentially function as a powerful predictor for liver cancer. The “M-score” calculated in a computing manner in the present invention has proven to be effective in predicting liver cancer at the very early stage. The M-score exceeds the predictive capabilities of serum markers.

Having described at least one of the embodiments of the claimed invention with reference to the accompanying drawings, it will be apparent to those skills that the invention is not limited to those precise embodiments, and that various modifications and variations can be made in the presently disclosed system without departing from the scope or spirit of the invention. Thus, it is intended that the present disclosure covers modifications and variations of this disclosure provided they come within the scope of the appended claims and their equivalents. Specifically, one or more limitations recited throughout the specification can be combined in any level of detail to the extent they are described to carry out the noninvasive liver cancer occurrence detecting method.

What is claimed is:

1. A method of detecting early-stage hepatocellular carcinoma, comprising:
performing biomarker identification of a group of differentially methylated genes in a computing system designed to process, analyze, simulate, and model biological data and equipped with a microprocessor by respectively detecting methylation level of the group of differentially methylated genes, the group of differentially methylated genes consisting of APC gene, COX2 gene, miR-203, RASSF1A gene, VIM gene, RGS10 gene, ST8SIA6 gene, and miR-129-2;
performing quantitative measurement of the methylation levels of a plurality of biomarkers selected from the group of differentially methylated genes with quantitative methylation-specific PCR (qMSP) in the computing system, wherein the selected biomarkers are the APC gene, COX2 gene, miR-203, RASSF1A gene, VIM gene, RGS10 gene, ST8SIA6 gene, and miR-129-2;
performing calculation of a formula in the computing system to obtain M-score of the selected biomarkers according to the measured methylation levels of the selected biomarkers with a logistic regression analysis, wherein the formula is M-score=X1+X2×ln(APC)+X3×ln(COX2)+X4×ln(miR-203)+X5×ln(RASSF1A)+X6×ln(VIM)+X7×ln(RGS10)+X8×ln(ST8SIA6)+X9×ln(miR-129-2), X1 ranges from 3.701 to 4.914, X2 ranges from 0.117 to 0.229, X3 ranges from 0.104 to 0.176, X4 ranges from 0.114 to 0.254, X5 ranges from 0.125 to 0.237, X6 ranges from 0.213 to 0.317, X7 ranges from 0.075 to 1.087, X8 ranges from 0.085 to 0.108, X9 ranges from 0.047 to 0.086, ln(APC) represents a hyperbolic logarithm of the methylation level of the APC gene, ln(COX2) represents a hyperbolic logarithm of the methylation level of the COX2 gene, ln(miR-203) represents a hyperbolic logarithm of the methylation level of the miR-203, ln(RASSF1A) represents a hyperbolic logarithm of the methylation level of the RASSF1A gene, ln(VIM) represents a hyperbolic logarithm of the methylation level of the VIM gene, ln(RGS10) represents a hyperbolic logarithm of the methylation level of the RGS10 gene, ln(ST8SIA6) represents a hyperbolic logarithm of the methylation level of the ST8SIA6 gene, and ln(miR-129-2) represents a hyperbolic logarithm of the methylation level of the miR-129-2; and
performing a risk level evaluation of liver cancer with the M-score of the selected biomarkers or a combination of AFP and the M-score of the selected biomarkers in the computing system.

2. The method of claim 1, further comprising:
performing a receiver operating characteristic (ROC) curve analysis of the M-score of the selected biomarkers or the combination of AFP and the M-score of the selected biomarkers in the computing system.

3. The method of claim 1, wherein the step of performing the quantitative measurement of the methylation levels of the selected biomarkers with the quantitative methylation-specific PCR (qMSP) in the computing system comprises performing a calculation of a difference in Ct value between housekeeping gene and each of the selected biomarkers and a formula 2^{[Ct(housekeeping gene)-Ct(biomarker)]}×100 is used to perform the calculation, and the Ct value is of single data points derived from real-time PCR amplification plots.

4. The method of claim 3, wherein the housekeeping gene is a gene selected from a group consisting of β-actin, GAPDH, HPRT, YWHAZ, ARBP, SDHA and UBC.

5. The method of claim 1, wherein the step of performing the quantitative measurement of the methylation levels of the selected biomarkers with the quantitative methylation-specific PCR (qMSP) in the computing system comprises using a kit to detect, the kit comprising:
a plurality of primer and probe sets targeting the APC gene, COX2 gene, miR-203, RASSF1A gene, VIM gene, RGS10 gene, ST8SIA6 gene, and miR-129-2; and
a qMSP master mix having Taq DNA polymerase, dNTPs, MgCl2 and buffer.

6. The method of claim 5, wherein each of the primer and probe sets includes a sense primer, an antisense primer, and a probe separately having a sequence correspondingly associated with the targeted gene.

7. The method of claim 1, wherein the methylation level of the APC gene is calculated using formula 2^{[Ct(β-actin)-Ct(APC)]}×100, the methylation level of the COX2 gene is calculated using formula 2^{[Ct(β-actin)-Ct(Cox2)]}×100, the methylation level of the MIR-203 is calculated using formula 2^{[Ct(β-actin)-Ct(MIR-203)]}×100, the methylation level of the RASSF1A gene is calculated using formula 2^{[Ct(β-actin)-Ct(RASSF1A)]}×100, the methylation level of the VIM gene is calculated using formula 2^{[Ct(β-actin)-Ct(VIM)]}×100, the methylation level of the RGS10 gene is calculated using formula 2^{[Ct(β-actin)-Ct(RGS10)]}×100, the methylation level of the ST8SIA6 gene is calculated using formula 2^{[Ct(δ-actin)-Ct(ST8SIA6)]}×100, and the methylation level of the miR-129-2 is calculated using formula 2^{[Ct(β-actin)-Ct(miR-129-2)]}×100, wherein Ct is of single data points derived from real-time PCR amplification plots.

8. A kit used in the method of claim 1 comprising:
a primer-pair and a probe for detecting the methylation level of the APC gene;
a primer-pair and a probe for detecting the methylation level of the COX2 gene;
a primer-pair and a probe for detecting the methylation level of the miR-203 gene;
a primer-pair and a probe for detecting the methylation level of the RASSF1A gene;
a primer-pair and a probe for detecting the methylation level of the VIM gene;
a primer-pair and a probe for detecting the methylation level of the RGS10 gene;
a primer-pair and a probe for detecting the methylation level of the ST8SIA6 gene; and
a primer-pair and a probe for detecting the methylation level of the miR-129-2 gene.
